(19) United States
(12) Patent Application Publication
(10) Pub. No.: US 2015/0111112 A1
(43) Pub. Date: Apr. 23, 2015

(54) FLUORINE-CONTAINING ESTERS AND METHODS OF PREPARATION THEREOF

(71) Applicant: E. I. DU PONT DE NEMOURS AND COMPANY, Wilmington, DE (US)

(72) Inventors: Viacheslav A. Petrov, Hockessin, DE (US); Charles J. Dubois, Wilmington, DE (US)

(21) Appl. No.: 14/404,246

(22) PCT Filed: Mar. 8, 2013
(86) PCT No.: PCT/US2013/029818

§ 371 Date: Nov. 26, 2014

Related U.S. Application Data
(60) Provisional application No. 61/654,524, filed on Jun. 1, 2012.
(60) Provisional application No. 61/654,524, filed on Jun. 1, 2012.

(51) Int. Cl. C07C 69/63; H01M 10/058; H01M 10/0567; C07C 67/10; H01M 10/0569

(52) U.S. Cl. CPC C07C 69/63; C07C 67/10; H01M 10/0569; H01M 10/0567; H01M 10/058; H01M 2300/0028; H01M 10/0525
USPC 429/343; 560/227; 29/623.1

(57) ABSTRACT

A method for preparing fluorine-containing carboxylic acid esters is described in which a salt of a carboxylic acid is reacted with a fluorinated alkyl halide. The fluorine-containing carboxylic acid esters prepared by the method disclosed herein are particularly useful as electrolyte solvents for electrochemical cells, such as a lithium ion battery, where a high purity solvent is desired.

This application claims priority under 35 U.S.C. 119(e) from, and claims the benefit of, U.S. Provisional Application No. 61/654,514 filed Jun. 1, 2012, and U.S. Provisional Application No. 61/654,524, filed Jun. 1, 2012, each of which is by this reference incorporated in its entirety as a part hereof for all purposes.

### TECHNICAL FIELD

The disclosure hereof relates to the field of organic synthesis. Specifically, this disclosure provides fluorine-containing carboxylic acid esters and methods of preparation thereof.

### BACKGROUND

Fluorine-containing carboxylic acid esters have many uses including use as an electrolyte solvent in electrochemical cells, such as lithium ion batteries. Fluorine-containing carboxylic acid esters can be produced using several different methods from various starting materials. One such method is the reaction of a metal carboxylate with a fluorinated alkyl halide. This reaction, however, can involve the use of an added catalyst or promoter, which can result in the presence of unwanted impurities in the final product.

For example, WO 2009/040367 (Weisenhofer) describes the synthesis of a fluorine-containing carboxylic acid ester using the reaction of a metal carboxylate with a fluorinated alkyl halide, with sodium iodide added as a catalyst or promoter. The disadvantage of the described method is that it results in the formation of iodide and/or iodine impurities, which are difficult to remove from the final product. For use as an electrolyte solvent in an electrochemical cell such as a lithium ion battery, fluorine-containing carboxy is acid esters of hi purity are desired.

A need thus remains for fluorine-containing carboxylic acid esters of desirably high levels of purity, and for methods of making those esters that do not result in the formation of impurities in the final product that are difficult or expensive to remove, such as iodide and/or iodine.

### SUMMARY

In one embodiment, there is provided herein a method of preparing an ester comprising the steps of:

(a) providing a salt of a carboxylic acid represented by the formula:

R¹COO⁻M⁺

wherein R¹ is a C₁ to C₁₀ alkyl group and M⁺ is selected from the group consisting of lithium, sodium, potassium and cesium ion;

(b) providing a fluorinated alkyl halide represented by the formula:

CF₂H—R²—X

wherein R² is a C₁ to C₁₀ alkylene group and X is selected from the group consisting of Br and Cl;

(c) contacting the salt of (a) with the alkyl halide of (b) in a reaction medium comprising a polar, aprotic solvent wherein X is displaced from the alkyl halide by the carboxylate anion of the salt (a) to form, an ester product; and

(d) optionally, recovering the ester product from the reaction medium.

In another embodiment, there is provided a method of preparing an ester comprising the steps of:

(a) providing a salt of a carboxylic acid represented by the formula:

R¹COO⁻M⁺

wherein R¹ is a C₁ to C₁₀ alkyl group and M⁺ is selected from the group consisting of lithium, sodium, potassium and cesium ion;

(b) providing a fluorinated alkyl halide represented by the formula:

CF₂H—R²—X

wherein R² is a C₁ to C₁₀ alkylene group and X selected from the group consisting of Brand Cl;

(c) contacting the salt of (a) with the alkyl halide of (b) in a reaction medium comprising a polar, aprotic solvent in the absence of any substance that participates in the formation of an intermediate or reactive substrate from the alkyl halide of (b) to form a product that comprises a single ester group; and

(d) optionally, recovering the ester product from the reaction medium.

In a further embodiment, there is provided a method of preparing an ester comprising the steps of:

(a) providing a salt of a carboxylic acid represented by the formula:

R¹COO⁻M⁺

wherein R¹ is a C₁ to C₁₀ alkyl group and M⁺ is selected from the group consisting of lithium, sodium, potassium and cesium ion;

(b) providing a fluorinated alkyl halide represented by the formula:

CF₂H—R²—X

wherein R² is a C₁ to C₁₀ alkylene group and X is selected from the group consisting of Br and Cl;

(c) contacting the salt of (a) with the alkyl halide of (b) in a reaction medium that comprises a polar, aprotic solvent and that is substantially free of iodine or iodide ion to form a product that comprises a single ester group; and

(d) optionally, recovering the ester product from the reaction medium.

In yet another embodiment, there is provided herein a method of preparing an ester comprising the steps of:

(a) providing a salt of a carboxylic acid represented by the formula:

R¹COO⁻M⁺

wherein R² is a C₁ to C₁₀ alkyl group and M⁺ is selected from the group consisting of lithium, sodium, potassium and cesium ion;

(b) providing a fluorinated alkyl halide represented by the formula:

CF₂H—R²—X

wherein R² is a C₁ to C₁₀ alkylene group;

contacting the salt of (a) with the alkyl halide of (b) in a reaction medium that comprises a polar, aprotic solvent and that is substantially free of iodine, iodide ion, chlorine or chloride ion to form a product that comprises a single ester group; and

(d) optionally, recovering the ester product from the reaction medium.

In yet other embodiments, there is provided herein a method comprising the steps of (a) providing a salt of a carboxylic acid represented by the formula:

R1COO−M+

wherein R1 is a C1 to C10 alkyl and M+ is at least one of sodium, potassium, or cesium ion; (b) providing a fluorinated alkyl halide represented by the formula:

HCF2-R2-X

wherein R2 is a C1 to C10 alkylene group and X is Cl and Br; (c) contacting the salt of the carboxylic acid of (a) with the fluorinated alkyl halide or (b) in a reaction medium comprising a polar, aprotic solvent to form a fluorine-containing carboxylic acid ester, wherein the reaction medium does not contain a deliberately added catalyst or promoter; and (d) optionally, recovering the fluorine-containing carboxylic acid ester from the reaction medium.

### DETAILED DESCRIPTION

Disclosed herein are fluorine-containing carboxylic acid esters, and methods for the preparation thereof. The methods disclosed herein do not cause the formation in the final ester product of undesirable levels of impurities, such as iodide (I⁻) and/or iodine (I₂).

The fluorine-containing carboxylic acid esters prepared by the methods disclosed herein are particularly useful as electrolyte solvents for electrochemical cells, such as a lithium ion battery, for which a high purity solvent is desired.

The methods disclosed herein can be used to prepare various fluorine-containing carboxylic acid esters, including without limitation those represented by the formula: R⁴—C(O)O—R⁵, where R4 and R5 independently represent an alkyl group, the sum of carbon atoms in R⁴ and R⁵ is 2 to 7, at least two hydrogens in R⁴ and/or R⁵ are replaced by fluorines and neither R4 nor R5 contains a —CH₂F or —CHF group. The presence of a monofluoroalkyl group (i.e. FCH₂ or FCH) in the carboxylic acid ester may cause toxicity. Suitable ester products thus include without limitation
CH₃—COO—CH₂CF₂H (2,2-difluoroethyl acetate),
CH₃CH₂—COO—CH₂CF₂H (2,2-difluoroethyl propionate),
CH₃—COO—CH₂CH₂CF₂H (3,3-difluoropropyl acetate),
CH₃CH₂—COO—CH₂CH₂CF₂H (3,3-difluoropropyl propionate), and (ethyl 4,4-difluorobutanoate).

The methods disclosed herein can also be used to prepare various fluorine-containing carboxylic acid esters, including without limitation
2,2-difluoroethyl butanoate [CH₃CH₂CH₂—C(O)O—CH₂CHF₂], or
2,2-difluoroethyl pentanoate [CH₃CH₂CH₂CH₂—C(O)O—CH₂CHF₂].

In certain embodiments hereof, the carboxylic acid ester prepared by the methods hereof contains a single ester group.

In the methods disclosed herein, the salt of the carboxylic acid is represented by the formula: (R¹COO⁻)ₙM⁺ⁿ, wherein R¹ is a C₁ to C₁₀ alkyl group and M⁺ⁿ is a cation other than hydrogen and n=1 or 2. The cation may be an alkali metal cation, and alkaline earth metal cation such as calcium or magnesium, an alkyl ammonium cation, or ammonium ion.

In some embodiments, the salt of the carboxylic acid is represented by the formula R¹COO⁻ wherein R¹ is a C₁ to C₁₀ alkyl group and M⁻ is at least one of sodium, potassium or cesium ion, or an alkyl ammonium ion [(R¹¹)(R¹²)(R¹³)(R¹⁴)N⁺] wherein each of R¹¹, R¹², R¹³ and R¹⁴ is independently H or a C₁˜C₅ alkyl group provided that at least one of them is not H. Preferred are tetraalkyl ammonium ions wherein none of R¹¹, R¹², R¹³ and R¹⁴ is H. Suitable salts of carboxylic acids include without limitation potassium acetate, potassium propionate, potassium butanoate, potassium pentanoate, sodium acetate, sodium propionate, sodium butanoate, sodium pentanoate cesium acetate, cesium propionate, cesium butanoate, or cesium pentanoate. Additionally, mixtures of these salts can also be used. For example, a mixture of potassium acetate and sodium acetate can be used.

The fluorinated alkyl compound used in the methods disclosed herein is represented by the formula: CHF₂—R²—X, wherein R² is a C₁ to C₁₀ alkylene group or fluoroalkylene group and X is a leaving group selected from the group consisting of Br, Cl, and —OSO₂R¹⁵ where R¹⁵ is aryl, F, CF₃, C₄F₉, alkyl or OC(O)X where X is Cl or F. The term “alkylene group” refers to a divalent group containing carbon and hydrogen, having only carbon-carbon single bonds, and which may be linear or branched. The term “fluoroalkylene” refers to an alkylene group wherein one or more hydrogens have been replaced by one or more fluorines. Although R² can contain fluorines, the group adjacent to X is CH₂.

In some embodiments, the fluorinated alkyl compound used in the methods disclosed herein is a fluorinated alkyl halide represented by the formula: CHF₂—R²—X, wherein R² is a C₁ to C₁₀ alkylene group or fluoroalkylene group and X is Cl, Br or I. Preferably, X is Cl or Br. Examples of useful fluorinated alkyl halides include without limitation CHF₂—CH₂—Br, CHF₂—CH₂—Cl, CHF₂—CH₂CH₂—Br, CHF₂—CH₂CH₂—Cl, CHF₂—CH₂CH₂CH₂—Br, and CHF₂—CH₂CH₂CH₂—Cl. In one particular embodiment, the fluorinated alkyl halide is CHF₂—CH₂—Br. In another particular embodiment, the fluorinated alkyl halide is CHF₂—CH₂—Cl. The fluorinated alkyl halides may be prepared using liquid phase or gas phase methods known in the art, for example using the methods described by Chen et al. (U.S. Patent Application Publication No. 2002/0183569), Bolmer et al. (U.S. Pat. No. 6,063,969), Boyce et al. (U.S. Pat. No. 5,910,616), or the method described in the Examples herein.

R¹ and R² can optionally contain fluorination themselves provided, as set forth above, that the presence of —CH₂F or —CHF groups does not result therefrom. Terminal CHF₂ and interior CF₂ groups separated from the reaction site by at least one carbon atom are preferred.

The salt of the carboxy is acid and the fluorinated alkyl compound, e.g., a fluorinated alkyl halide, are contacted in the absence of any substance that participates in the formation of an intermediate or reactive substrate from the alkyl halide (e.g., sodium iodide) to form a product that comprises a single ester group. In one embodiment, the salt of the carboxylic acid and the fluorinated alkyl compound are con acted in a reaction medium comprising a solvent. Suitable solvents, include without limitation, nitriles, dinitriles, such as adiponitrile, esters, including esters containing fluorine, and ethers such as diglyme, triglyme, and tetraglyme.

In some embodiments, the salt of the carboxylic acid and the fluorinated alkyl halide are contacted in a reaction medium comprising a polar, aprotic solvent to form the fluorine-containing carboxylic acid ester. A polar, aprotic solvent refers to a solvent having a high dielectric constant and a high dipole moment, but lacking an acidic hydrogen. Suitable polar, aprotic solvents can typically be selected from the substituted acid amides, the organic sulfoxides and the cyclic amides, and mixtures thereof. The substituted acid amides can be represented by the general formula:

R⁶—C(O)—N(R⁷)—R⁸

where R⁶ is selected from the group consisting of hydrogen and a hydrocarbon radical having between 1 and 8 carbon atoms; R⁷ and R⁸ are selected from the group consisting of hydrogen and an alkyl radical having between 1 and 3 carbon atoms, provided that R⁷ and R⁸ are not both hydrogen, and wherein the acid amide contains at least two carbon atoms. Examples of preferred acid amides include N-methylformamide, N,N-dimethylformamide, N,N-dimethylacetamide and N,N-dimethylpropionamide.

The organic sulfoxides can be represented by the general formula:

R⁹—S(O)—R¹⁰

where R⁹ and R¹⁰ can be the same or different and are hydrocarbon radicals having between 1 and 8 carbon atoms. Examples of suitable sulfoxides include dimethylsulfoxide, diethylsulfoxide, ethylpropylsulfoxide, dioctylsulfoxide, benzylmethylsulfoxide, diphenylsulfoxide, paramethylphenylethylsulfoxide, and dichloromethylsulfoxide. Dimethylsulfoxide is a preferred sulfoxide.

In other embodiments, suitable polar, aprotic solvents can be selected from the group consisting of sulfolane, N-methyl-2-pyrrolidone, N,N-dimethyl-2-imidazolidinone, 1,3-dimethyl-3,4,5,6-tetrahydro-2(1H)-pyrimidinone, and mixtures thereof.

The weight ratio of the solvent to the combined weights of the salt and alkyl halide reactants can be in the range of about 0.5/1 to about 50/1, or in the range of about 0.5/1 to about 20/1, or in the range of about 1/1 to about 10/1. The reaction may occur in a batch or in a continuously fed reactor in which one or both reactants and optionally solvent are fed on a continuous basis. Product may accumulate in the reactor or be removed on a continuous basis.

During the reaction, the temperature of the reaction medium is about 20° C. to about 200° C., more particularly about 50° C. to about 150° C., and more particularly about 80° C. to about 120° C. The reaction medium may be agitated during the reaction using conventional means such as a magnetic stirrer, an overhead mixer, and the like. In various embodiments, the reaction pressure can be maintained at a level at which the solvent and reactants are kept in the liquid phase. A pressure between atmospheric and 1,000 psig is suitable for such purpose.

The methods hereof involve contacting a salt of a carboxylic acid with a fluorinated alkyl halide in a reaction medium that does not contain a deliberately added catalyst such as sodium iodide or potassium iodide. Therefore, the reaction medium is substantially free of iodide and/or iodine.

Therefore, the reaction medium and the resulting fluorine-containing carboxylic acid ester are substantially free of iodide and/or iodine, although traces of iodide and/or iodine from impurities in the reactants and solvent may be present.

In the methods hereof, there is an absence of any substance that participates in the formation of an intermediate or a reactive substrate from the alkyl halide. Also, in the methods hereof, the reaction medium does not contain any deliberately added catalyst or promoter. The reaction in WO 2009/40367 is an example of the use of a substance that does participates in the formation of an intermediate or a reactive substrate from the alkyl halide since I— ion from the NaI compound displaces the Br from the alkyl halide before I itself is subsequently displaced from the alkyl halide by the acetate ion. NaI in that reaction thus is also an example of a deliberately added catalyst or promoter.

In other embodiments of the methods hereof, the reaction mixture is free or substantially free of one or more of iodine, iodide, bromide, and/or chloride. Preferably, the reaction mixture is free or substantially free of iodine and iodide. Substantially free is defined as an amount of less than about 10⁵, less than about 10⁴, less than about 10³, less than about 5×10², less than about 10², less than 10, or less than 1 ppm.

The fluorine-containing carboxylic acid ester formed in the reaction may optionally be isolated from the reaction medium and purified using methods known in the art, e.g. distillation methods such as vacuum distillation or spinning band distillation. For best results when used as an electrolyte solvent in a lithium ion battery, as discussed below, it is desirable to purify the fluorine-containing carboxylic acid esters to a purity level of at least about 99.9%, more particularly at least about 99.99%.

In the fluorinated esters hereof, that are produced by the methods hereof, the content of any one, any two, any three, any four, any five or all six of the following impurities: iodine, iodide, chloride, bromide, water and/or a fluorinated alcohol (such as 1,1-difluoroethanol) is less than about 10⁵, less than about 10⁴, less than about 10³, less than about 5×10², less than about 10², less than 10, or less than 1 ppm. Methods of purification are disclosed herein and known in the art.

In one embodiment, the fluorine-containing carboxylic acid ester prepared by the methods disclosed herein is admixed with at least one electrolyte salt to form an electrolyte composition. Suitable electrolyte salts include without limitation

lithium hexafluorophosphate (LiPF₆),

lithium tris(pentafluoroethyl)trifluorophosphate (LiPF₃ (C₂F₅)₃),

lithium bis(trifluoromethanesulfonyl)imide,

lithium bis(perfluoroethanesulfonyl)imide,

lithium(fluorosulfonyl)(nonafluorobutanesulfonyl)imide,

lithium bis(fluorosulfonyl)imide,

lithium tetrafluoroborate,

lithium perchlorate,

lithium hexafluoroarsenate,

lithium trifluoromethanesulfonate,

lithium tris(trifluoromethanesulfonyl)methide,

lithium bis(oxalato)borate,

lithium difluoro(oxalato)borate,

Li₂B₁₂F₁₂₋ₓHₓ where x is equal to 0 to 8, and

mixtures of lithium fluoride and anion receptors such as B(OC₆F₅)₃.

Mixtures of two or more of these or comparable electrolyte salts may also be used. In one embodiment, the electrolyte salt is lithium hexafluorophosphate. The electrolyte salt can be present in the electrolyte composition in an amount of about 0.2 to about 2.0 M, more particularly about 0.3 to about 1.5 N, and more particularly about 0.5 to about 1.2 M.

The electrolyte composition may also contain at least one co-solvent, which is added to the composition along with the fluorine-containing carboxylic acid ester prepared by the methods hereof. Examples of suitable co-solvents include without limitation various carbonates and sulfones. Suitable co-solvents include without limitation ethylmethyl carbonate, diethyl carbonate, ethylene carbonate, propylene carbonate, fluoroethylene carbonate, tetramethylene sulfone and ethyl methyl sulfone. For best results, it is desirable to use a co-solvent that is battery grade or has a purity level of at least about 99.9%, and more particularly at least about 99.99%. In one embodiment, the co-solvent is ethylene carbonate.

The fluorine-containing carboxylic acid ester, prepared by the methods disclosed herein, and the co-solvent may be combined in various ratios to form a solvent mixture as used in the electrolyte composition, depending on the desired properties of the electrolyte composition. In one embodiment, the fluorine-containing carboxylic acid ester comprises about 10% to about 90% by weight of the solvent mixture. In another embodiment, the fluorine-containing carboxylic acid ester comprises about 40% to about 90% by weight of the solvent mixture. In another embodiment, the fluorine-containing carboxylic acid ester comprises about 50% to about 80% by weight of the solvent mixture. In another embodiment, the fluorine-containing carboxylic acid ester comprises about 60% to about 80% by weight of the solvent mixture. In another embodiment, the fluorine-containing carboxylic acid ester comprises about 65% to about 75% by weight of the solvent mixture. In another embodiment, the fluorine-containing carboxylic acid ester comprises about 70% by weight of the solvent mixture.

The electrolyte composition can be contacted with a cathode and an anode to form an electrochemical cell, such as a lithium ion battery. A cathode the electrode of an electrochemical cell at which reduction occurs. In a galvanic cell such as a battery, the cathode is the positively charged electrode. In a secondary (i.e. rechargeable) battery, the cathode is the electrode at which reduction occurs during discharge and oxidation occurs during charging. An anode is the electrode of an electrochemical cell at which oxidation occurs. In a galvanic cell, such as a battery, the anode is the negatively charged electrode. In a secondary (i.e. rechargeable) battery, the anode is the electrode at which oxidation occurs during discharge and reduction occurs during charging. The fluorine-containing carboxylic acid esters prepared by the method disclosed herein are particularly useful for use in electrochemical cells, such as lithium ion batteries, wherein high purity solvents are desired, because the fluorine-containing carboxylic acid esters are substantially free of impurities such as iodide and/or iodine.

An electrochemical cell comprises a housing, an anode and a cathode disposed in the housing and in ionically conductive contact with one another, an electrolyte composition, as described above, providing an ionically conductive pathway between the anode and the cathode, and a porous or microporous separator between the anode and the cathode. The housing may be any suitable container to house the electrochemical cell components. The anode and the cathode may be comprised of any suitable conducting material depending on the type of electrochemical cell. Suitable examples of anode materials include without limitation lithium metal lithium metal alloys, lithium titanate, aluminum, platinum, palladium, graphite, transition metal oxides, and lithiated tin oxide. Suitable examples of cathode materials include without limitation graphite, aluminum, platinum, palladium, electroactive transition metal oxides comprising lithium or sodium, indium tin oxide, and conducting polymers such as polypyrrole and polyvinylferrocene.

The porous separator serves to prevent short circuiting between the anode and the cathode. The porous separator typically consists of a single-ply or multi-ply sheet of a microporous polymer such as polyethylene, polypropylene, or a combination thereof. The pore size of the porous separator is sufficiently large to permit transport of ions, but small enough to prevent contact of the anode and cathode either directly or from particle penetration or dendrites which can from on the anode and cathode.

In one embodiment, the electrochemical cell is a lithium ion battery, which is a type of rechargeable battery in which lithium ions move from the anode to the cathode during discharge, and from the cathode to the anode during charge. Suitable cathode materials for a lithium ion battery include without limitation electroactive transition metal oxides comprising lithium, such as LiCoO₂, LiNiO₂, LiMn₂O₄ or LiV₃O₈.

Various lithium composite oxides containing lithium and a transition metal may be utilized as the cathode material. Suitable examples include composite oxides with the general formula LiMO₂ where M can be any metallic elements or combination of metallic elements such as cobalt, aluminum, chromium, manganese, nickel, iron, vanadium, magnesium, titanium, zirconium, niobium, molybdenum, copper, zinc, indium, strontium, lanthanum, and cesium. Additionally, the active material can be made of a material with the chemical formula LiMn₂₋ₓMₓO₄, where 0≦x≦1,or a material with the general formula LiMPO₄ where M can be any metallic element or combination of elements such as cobalt, aluminum, chromium, manganese, nickel, iron, vanadium, magnesium, titanium, zirconium, niobium, molybdenum, copper, zinc, indium, strontium, lanthanum, and cesium. The cathode of the battery may include any of the active materials that may be held on an electrical conductive member that includes metal or another conductive element.

In one embodiment, the cathode in the lithium ion battery hereof comprises a cathode active material exhibiting greater than 30 mAh/g capacity in the potential range greater than 4.6 V versus a Li/Li⁺ reference electrode. One example of such a cathode is a stabilized manganese cathode comprising a lithium-containing manganese composite oxide having a spinel structure as cathode active material. The lithium-containing manganese composite oxide in a cathode as used herein comprises oxides of the formula LiₓNiₓM_{z}Mn_{2-y-z}O_{4-c}, wherein x is 0.03 to 1.0; x changes in accordance with release and uptake of lithium ions and electrons during charge and discharge; y is 0.3 to 0.6; M comprises one or more of Cr, Fe, Co, Al, Ga, Nb, Mo, Ti, Zr, Mg, Zn, V, and Cu; z is 0.01 to 0.18, and d is 0 to 0.3. In one embodiment, in the above formula, y is 0.38 to 0.48, z is 0.03 to 0.12, and d is 0 to 0.1. In one embodiment, in the above formula, M is one or more of Li, Cr, Fe, Co, and Ga. Stabilized manganese cathodes may also comprise spinel-layered composites which contain a manganese-containing spinel component and a lithium rich layered structure, as described in U.S. Pat. No. 7,303,840.

The cathode active material can be prepared using methods such as the hydroxide precursor method described by Liu et al (J. Phys. Chem., C 13:15073-15079, 2009). In that method, hydroxide precursors are precipitated from a solution containing the required amounts of manganese, nickel and other desired metal (s) acetates by the addition of KOH. The resulting precipitate is oven-dried and then fired with the required amount of LiOH.H₂O at about 800 to about 950° C. in oxygen for 3 to 24 hours, as described in detail in the examples herein. Alternatively, the cathode active material can be prepared using a solid phase reaction process or a sol-gel process as described in U.S. Pat. No. 5,738,957 (Amine).

The cathode, in which the cathode active material is contained, may be prepared by methods such as mixing an effective amount of the cathode active material (e.g. about 70 wt % to about 97 wt %), a polymer binder, such as polyvinylidene difluoride, and conductive carbon in a suitable solvent, such as N-methylpyrrolidone, to generate a paste, which is then coated onto a current collector such as aluminum foil, and dried to form the cathode.

The lithium ion battery hereof further contains an anode, which comprises an anode active material that is capable of storing and releasing lithium ions. Examples of suitable anode active materials include without limitation lithium alloys such as lithium-aluminum alloy, lithium-lead alloy, lithium-silicon alloy, lithium-tin alloy and the like; carbon materials such as graphite and mesocarbon microbeads (MCMB); phosphorus-containing materials such as black phosphorus, MnP₄ and CoP₃; metal oxides such as SnO₂, SnO and TiO₂; and lithium titanates such as Li₄Ti₅O₁₂ and LiTi₂O₄. In one embodiment, the anode active material is lithium titanate or graphite.

An anode can be made by a method similar to that described above for a cathode wherein, for example, a binder such as a vinyl fluoride-based copolymer is dissolved or dispersed in an organic solvent or water, which is then mixed with the active, conductive material to obtain a paste. The paste is coated onto a metal foil, preferably aluminum or copper foil, to be used as the current collector. The paste is dried, preferably with heat, so that the active mass is bonded to the current collector. Suitable anode active materials and anodes are available commercially from companies such as Hitachi NEI Inc. (Somerset, N.J.), and Farasis Energy Inc. (Hayward, Calif.).

The lithium ion battery hereof also contains a porous separator between the anode and cathode. The porous separator serves to prevent short circuiting between the anode and the cathode. The porous separator typically consists of a single-ply or multi-ply sheet of a microporous polymer such as polyethylene, polypropylene, polyamide or polyimide, or a combination thereof. The pore size of the porous separator is sufficiently large to permit transport of ions to provide ionically conductive contact between the anode and cathode, but small enough to prevent contact of the anode and cathode either directly or from particle penetration or dendrites which can from on the anode and cathode. Examples of porous separators suitable for use herein are disclosed in U.S. application Ser. No. 12/963,927 (filed 9 Dec. 2010, U.S. Patent Application Publication No. 2012/0149852), which is by this reference incorporated in its entirety as a part hereof for all purposes.

The housing of the lithium ion battery hereof may be any suitable container to house the lithium ion battery components described above. Such a container may be fabricated in the shape of small or large cylinder, a prismatic case or a pouch.

The lithium ion battery hereof may be used for grid storage or as a power source in various electronically powered or assisted devices (an “Electronic Device”) such as a transportation device (including a motor vehicle, automobile, truck, bus or airplane), a computer, a telecommunications device, a camera, a radio, or a power tool.

### EXAMPLES

The subject matter disclosed herein is further defined in the following examples. It should be understood that these examples, while indicating preferred embodiments of some the inventions hereof, are given by way of illustration only, and should not be interpreted to exclude from the scope of the appended claims, and the equivalents thereof, subject matter that is not described in these examples.

The meaning of abbreviations used is as follows: means gram(s), “mg” means milligram(s), “μg” means microgram(s), “L” means liter(s), “mL” means milliliter(s), “mol” means mole(s), “mmol” means millimole(s), “M” means molar concentration, “wt %” means percent by weight, “mm” means millimeter (s), “ppm” means parts per million, “h” means hour (s), “min” means minute (s), “Hz” means hertz, “mS” means millisiemen(s), “mA” mean milliamp(s), “mAh/g” mean milliamp hour (s) per gram, “V” means volt(s), “xC” refers to a constant current that can fully charge/discharge the cathode in 1/x hours, “SOC” means state of charge, “SEI” means solid electrolyte interface formed on the surface of the electrode material, “Pa” means pascal(s), “kPa” means kilopascal(s), “rpm” means revolutions per minute, “psi” means pounds per square inch, “NMR” means nuclear magnetic resonance spectroscopy, “GC/MS” means gas chromatography/mass spectrometry, “b.p.” means boiling point.

### Example 1

### Preparation of 2,2-Difluoroethyl Acetate

Potassium acetate (Aldrich, Milwaukee, Wis., 99%) was dried at 100° C. under a vacuum of 0.5-1 mm of Hg (66.7-133 Pa) for 4 to 5 h. The dried material had a water content of less than 5 ppm, as determined by Kari Fischer titration, In a dry box, 212 g (2.16 mol, 8 mol % excess) of the dried potassium acetate was placed into a 1.0-L, 3 neck round bottom flask containing a heavy magnetic stir bar. The flask was removed from the dry box, transferred into a fume hood, and equipped with a thermocouple well, a dry-ice condenser, and an additional funnel.

Sulfolane (500 mL, Aldrich, 99%, 600 ppm of water as determined by Karl Fischer titration) was melted and added to the 3 neck round bottom flask as a liquid under a flow of nitrogen. Agitation was started and the temperature of the reaction medium was brought to about 100° C. HCF₂CH₂Br (290 g, 2 mol, E.I. du Pont de Nemours and Co., 99%) was placed in the addition funnel and was slowly added to the reaction medium. The addition was mildly exothermic and the temperature of the reaction medium rose to 120-130° C. in 15-20 min after the start of the addition. The addition of HCF₂CH₂Br was kept at a rate which maintained the internal temperature at 125-135° C. The addition took about 2-3 h. The reaction medium was agitated at 120-130° C. for an additional 6 h (typically the conversion of bromide at this point was about 90-95%). Then, the reaction medium was cooled down to room temperature and was agitated overnight. Next morning, heating was resumed for another 8 h.

At this point the starting bromide was not detectable by NMR and the crude reaction medium contained 0.2-0.5% of 1,1-difluoroethanol. The dry-ice condenser on the reaction flask was replaced by a hose adapter with a Teflon® valve and the flask was connected to a mechanical vacuum pump through a cold trap (−78° C., dry-ice/acetone). The reaction product was transferred into the cold trap at 40-50° C., under a vacuum of 1-2 mm Hg (133 to 266 Pa). The transfer took about 4-5 h and resulted in 220-240 g of crude HCF₂CH₂OC(O)CH₃ of about 98-98.5% purity, which was contaminated by a small amount of HCF₂CH₂Br (about 0.1-0.2%), HCF₂CH₂OH (0.2-0.8%), sulfolane (about 0.3-0.5%) and water (600-800 ppm). Further purification of the crude product was carried out using spinning band distillation at atmospheric pressure. The fraction having a boiling point between 106.5-106.7° C. was collected and the impurity profile was monitored using GC/MS (capillary column HP5MS, phenyl-methyl siloxane, Agilent19091S-433, 30.m, 250 μm, 0.25 μm; carrier gas—He, flow rate 1 mL/min; temperature program: 40° C., 4 min, temp. ramp 30° C./min, 230° C., 20 min). Typically, the distillation of 240 g of crude product gave about 120 g of HCF₂CH₂OC(O)CH₃ of 99.89% purity, (250-300 ppm H₂O) and 80 g of material of 99.91% purity (containing about 280 ppm of water). Water was removed from the distilled product by treatment with 3A molecular sieves, until water was not detectable by Karl Fischer titration (i.e., <1 ppm).

### Example 2

### Preparation of 2,2-Difluoroethyl Propionate

Potassium propionate (Aldrich, 99%) was dried at 100° C. under a vacuum of 0.5-1 mm of Hg (66.7-133 Pa) for 4 to 5 h. The dried material had a water content of less than 5 ppm, as determined by Karl Fischer titration. In a dry box, 75 g (0.67 mol, 10 mol % excess) of the dried potassium propionate was placed into a 500 mL, 3 neck round bottom flask containing a heavy magnetic stir bar. The flask was removed from the dry box, transferred into a fume hood, and equipped with a thermocouple well, a dry ice condenser, and an additional funnel. Sulfolane (300 mL, Aldrich 99%, 600 ppm of water as determined by Karl Fischer titration) was melted and added to the 3 neck round bottom flask as a liquid under a flow of nitrogen. Agitation was started and the temperature of the reaction medium was brought to about 100° C. HCF₂CH₂Br (87 g, 0.6 mol, E.I. du. Pont de Nemours and Co., 99%) was placed in the addition funnel and was slowly added to the reaction medium. The addition was mildly exothermic and the temperature rose to 120-130° C. in 15-20 min after the start of the addition. The addition or HCF₂CH₂Br was kept at a rate which maintained the internal temperature at 125-135° C. The addition took about 2-3 h. The reaction medium was agitated at 120-130° C. for an additional 6 h(typically the conversion of bromide at this point was about 90-95%). Then, the reaction medium was cooled down to room temperature and was agitated overnight. Next morning, heating was resumed for another 8 h.

At this point, the starting bromide and 1,1-difluoroethanol were not detectable in the crude reaction medium by NMR. The dry-ice condenser on the reaction flask was replaced by a hose adapter with a Teflon® valve and the flask was connected to a mechanical vacuum pump through a cold trap (−78° C., dry-ice/acetone). The reaction product was transferred into the cold trap at 40-50° C. under a vacuum of 1-2 mm Hg (133 to 266 Pa). The transfer took about 3 h and resulted in 48 g of crude HCF₂CH₂OC(O)C₂H₅ of about 98% purity. Further purification of the crude product was carried out using spinning band distillation at atmospheric pressure. The fraction having a boiling point between 120.3-120.6° C. was collected and the impurity profile was monitored using GC/MS (capillary column HP5MS, phenyl-methyl siloxane, Agilent 19091S-433, 30 m, 250 μm, 0.25 μm; carrier gas—He, flow rate 1 mL/min; temperature program: 40° C., 4 min, temp. ramp 30° C./min. 230° C., 20 min). The crude product (43 g) had a purity of 99.91% and contained about 300 ppm of water. Water was removed from the product by treatment with 3A molecular sieves, until water was not detectable by Karl Fischer titration (i.e., <1 ppm).

HCF₂CH₂OC(O)C₂H₅: 1H NMR (CDCl₃): 1.10 (3H.t), 2.35 (2H, q), 4.21 (2H, td), 5.87 (1H, tt) ppm; ¹⁹F NMR (CDCl3): −125.68 (dt, 56.6, 13.7 Hz) ppm, GS/MS(m/z): 138(M+, C₅H₈F₂O₂+).

### Example 3

### Preparation of Nonaqueous Electrolyte Composition Comprising 2,2-Difluoroethyl Acetate (DFEA) and Ethylene Carbonate (EC)

2,2-Difluoroethyl acetate, prepared as described above, was purified by spinning band distillation twice to 99.992% purity, as determined by gas chromatography using a mass spectrometric detector. The purified 2,2-difluoroethyl acetate and ethylene carbonate (anhydrous, Novolyte, Independence, Ohio) were mixed together to make 15 mL of total solution in a 70:30 w/w ratio, and the resulting mixture was dried over 3A molecular sieves (Sigma-Aldrich, Milwaukee, Wis.). After drying, the water content was determined to be <0.5 ppm using Karl Fischer titration. The solution was syringe filtered through a 0.2 μm PTFE syringe filter. To 15.0 mL of the resulting solution was added 2.28 g of lithium hexafluorophosphate (battery grade, Novolyte) and the mixture was shaken for a few minutes until all the solid was dissolved.

### Example 4

### Preparation of Nonaqueous Electrolyte Composition Comprising 2,2-Difluoroethyl Propionate (DFEP)

2,2-Difluoroethyl propionate, prepared as described above, was purified by spinning hand distillation twice to 99.990% purity, as determined by gas chromatography using a mass spectrometric detector. The purified 2,2-difluoroethyl propionate was dried over 3A molecular sieves (Sigma-Aldrich, Milwaukee, Wis.). After drying, the water content was determined to be <0.5 ppm using Karl Fischer titration. The solution was syringe filtered through a 0.2 μm PTFE syringe filter. To the resulting DEEP (7.0 mL) was added a sufficient amount of lithium hexafluorophosphate (battery grade, Novolyte) to give a concentration of 1.0 M. The mixture was shaken for a few minutes until all the solid was dissolved.

### Example 5

### Preparation of Nonaqueous Electrolyte Composition Comprising 2,2-Difluoroethyl Propionate (DFEP) and Ethylene Carbonate (EC)

2,2-Difluoroethyl propionate, prepared as described above, was purified by spinning band distillation twice to 99.990% purity, as determined by gas chromatography using a mass spectrometric detector. The purified 2,2-difluoroethyl acetate and ethylene carbonate (anhydrous, Novolyte, independence, OH) were mixed together to make 9.0 mL of total solution in a 70:30 w/w ratio, and the resulting mixture was dried over 3A molecular sieves (Sigma-Aldrich, Milwaukee, Wis.). After drying, the water content was determined to be <0.5 ppm using Karl Fischer titration. The solution was syringe filtered through a 0.2 μm PTFE syringe filter. To 9.0 mL of the resulting solution was added lithium hexafluorophosphate (battery grade, Novolyte) to give a concentration of 1.0 M. The mixture was shaken for a few minutes until all the solid was dissolved.

### Examples 6-8

### Use of Electrolyte Compositions Containing 2,2-Difluoroethyl Acetate or 2,2-Difluoroethyl Propionate in Full Cells

### Preparation of LiMn1.5Ni0.42Fe0.08O4 Cathode Active Material

For LiMn_{1.5}Ni_{0.42}Fe_{0.08}O₄, 401 g manganese (II) acetate tetrahydrate (Aldrich 63537), 115 g nickel (II) acetate tetrahydrate (Aldrich 72225) and 15.2 g iron (II) acetate anhydrous (Alfa Aesar 31140) were weighed into bottles on a balance then dissolved in 5 L of deionized water. KOH pellets were dissolved in 10 L of deionized water to produce a 3.0 M solution inside a 30 L reactor. The acetate solution was transferred to an addition funnel and dripped into the rapidly stirred reactor to precipitate the mixed hydroxide material. Once all 5 L of the acetate solution was added to the reactor stirring was continued for 1 h. Then stirring was stopped and the precipitate was allowed to settle overnight. After settling the liquid was removed from the reactor and 15 L of fresh deionized water was added. The contents of the reactor were stirred, allowed to settle again, and liquid removed. This rinse process was repeated. Then the precipitate was transferred to two (split evenly) coarse glass frit filtration funnels covered with Dacron® paper. The solids were rinsed with deionized water until the filtrate pH reached 6 (pH of deionized rinse water), and a further 20 L of deionized water was added to each filter cake. Finally the cakes were dried in a vacuum oven at 120° C. overnight. The yield at this point was typically 80-90%.

The hydroxide precipitate was next ground and mixed with lithium carbonate. This step was done in 60 g batches using a Fritsche Puiverisette automated mortar and pestle. For each batch the hydroxide mixture was weighed, then ground alone for 5 minutes in the Puiveresette. Then a stoichiometric amount with small excess of lithium carbonate was added to the system. For 53 p of hydroxide 11.2 g of lithium carbonate was added. Grinding was continued for a total of 60 minutes with stops every 10-15 minutes to scrape the material off of the surfaces of the mortar and pestle with a sharp metal spatula. If humidity caused the material to form clumps, it was sieved through a 40 mesh screen once during grinding, then again following grinding.

The ground material was fired in air in a box furnace inside shallow rectangular alumina trays. The trays were 158 mm by 69 mm in size, and each held about 60 g of material. The firing procedure consisted of ramping from room temperature to 900° C. in 15 hours, holding at 900° C. for 12 hours, then cooling to room temperature in 15 hours.

### Preparation of an Iron-Doped, Lithium, Nickel, Manganese Oxide (Fe-LNMO) Cathode

The following is a description of a representative preparation of an Fe-LNMO cathode. The cathode active material LiMn_{1.5}HNi_{0.42}Fe_{0.08}O₄, prepared as describe above, was ground for ten minutes using an agate mortar and pestle and then passed through a 75 μm sieve. Particle size was measured to be 18 μm (d90). The sized cathode active material (1.240 g), 0.155 g of Denka black (acetylene black, DENKA Corp., Japan), 1.292 g of polyvinylidene difluoride (PVDF) solution (12 wt % in N-methylpyrrolidone (NMT), Kureha America Inc., New York, N.Y., KFL#1120), and an additional 2.313 g of anhydrous NMP (Sigma-Aldrich, Milwaukee, Wis.) were mixed first using a planetary centrifugal mixer (THINKY ARE-310, THINKY Corp., Japan) at 2,000 rpm, a shear mixer (VWR, Wilmington, N.C.), and then a planetary centrifugal mixer at 2,000 rpm to form a uniform slurry. The slurry was coated on 25 μm thick aluminum foil using a doctor blade, dried on a hot plate at 100° C. for five to seven minutes, then in a vacuum oven at 100° C. for five to seven minutes. The resulting 25-mm wide cathode was placed on a 125 μm thick brass sheet and two 38 mm wide brass shim strips of 87 μm thickness were placed on either side of the cathode to control the gap thickness in the calender. The electrode and shims were covered with a second 125 μm thick brass sheet, and the assembly was passed through a calender three times using 100 mm diameter steel rolls heated to 125° C. with a nip force of 154, 205, and 356 kg, respectively. The cathode was further dried in a vacuum oven at 90° C. at −25 inches Hg (−85 kPa) for 15 h.

### Preparation of a Lithium Titanate (LTO) Anode

The following is a description of a representative preparation of an LTO anode. The LTO anode active material, Li₄Ti₅O₁₂ (NEI Nanomyte™ BE-10, Somerset, N.J.), was ground for ten minutes using an agate mortar and pestle. The ground anode active material (3,168 g), 0.396 g of Super P Li carbon (Timcal. Switzerland), 3.300 g of polyvinylidene difluoride (PVDF) solution (12 wt % in N-methylpyrrolidone (NMT), Kureha America Inc., New York, N.Y., KFL#1120), and an additional 4.136 g of NMP were mixed first using a planetary centrifugal mixer (THINKY ARE-310, THINKY Corp., Japan) at 2,000 rpm, a shear mixer (VWR, Wilmington, N.C.), and then a planetary centrifugal mixer at 2,000 rpm to form a uniform slurry. The slurry was coated on copper foil using a doctor blade, and dried first on a hot plate at 100° C. for five to seven minutes, then a vacuum oven at 100° C. for five to seven minutes. The resulting electrode was calendered at 125° C. to constant thickness as previously described.

Fabrication of LTO|electrolyte|Fe-LNMO Full Cells

The following is a description of a representative preparation of full cells containing an Fe-LNMO cathode, an LTO anode and an electrolyte composition. Circular anodes 15 mm in diameter and cathodes 14 mm in diameter were punched out, placed in a heater in the antechamber of a glove box, further dried under vacuum at 90° C. for 15 h, and brought in to an argon glove box (Vacuum Atmospheres, Hawthorne, Calif., Nexus purifier). Nonaqueous electrolyte lithium-ion CR2032 coin cells were prepared for electrochemical evaluation. The coin cell parts (stainless steel case, two spacers, wave spring, lid, and polypropylene gasket) and coin cell crimper were obtained from Hohsen Corp (Osaka, Japan). An Fe-LNMO cathode, prepared as described above, a Celgard® separator 2325 (Celgard, LLC. Charlotte, N.C.), an LTO anode, prepared as described above, and a few drops of the nonaqueous electrolyte composition of interest, were assembled to form the LTO/Fe-LNMO full cells.

### High Temperature Performance of Full Cells

Full cell, containing the anode, cathode, and nonaqueous electrolyte shown in Table were cycled using a commercial battery tester (Series 4000, Maccor, Tulsa, Okla.) in a temperature-controlled chamber at 55° C. using voltage limits of 1.9 to 3.4 V. The constant-current charge and discharge currents for the first two cycles were 12 mA/g of LNMO (about 0.1 C rate), and subsequent cycles were carried out at 120 mA/g of LNMO for 29 cycles (about 1 C rate) then one cycle at 12 mA/g then repeated until T80 was reached. T80 defined as the number of cycles before the cell's discharge capacity has been reduced to 80% of the initial discharge capacity of the third charge-discharge cycle (first cycle at the 1 C rate).

TABLE 1
High Temperature Performance of Full Cells

| | | | Cycle |
|---|---|---|---|
| | Anode/ | | No. to |
| | Cathode | Electrolyte | T80 |
| Example 6 | LTO/ | DFEA + EC | 51 |
| | Fe-LNMO | | |
| Example 7 | LTO/ | DFEP | 75 |
| | Fe-LNMO | | |
| Example 8 | LTO/ | DFEP + EC | 87 |
| | Fe-LNMO | | |

### Example 9

### Preparation of 2,2-Difluoroethyl Acetate using HCF₂CH₂Cl in DMSO solvent

2,2-Difluoroethyl acetate was prepared by reacting potassium acetate with HCF₂CH₂Cl in DMSO.

### Preparation of HCF₂CH₂Cl

HCF₂CH₂Cl was prepared using a modification of the procedure described by V. Petrov et al. (Journal of Fluorine Chemistry, 125(2004), p. 103) as follows.

SOCl₂ (Fluka, Milwaukee, Wis., 99.9%, 75 mL) was placed in a 500-mL round bottom flask equipped with a magnetic stir bar, thermocouple, dry ice condenser, addition funnel and a connection to a water scrubber system to absorb evolving HCl. The flask was cooled down to 5° C., and the addition of HCF₂CH₂OH (62 g, 1 mol, 99%, SynQuest Laboratories, Alachua, Fla.) was started. The rate of addition was adjusted to allow the control of temperature and gas evolution. After the addition of the alcohol was finished (at 0-5° C.) the temperature of the reaction mixture was slowly raised to 35° C. The reaction mixture was kept at this temperature for 2 h and was agitated overnight at ambient temperature. Next morning the dry ice condenser was replaced with a hose connector and the flask was connected to a vacuum source through a cold trap (−78° C.). The flask was slowly evacuated to about 100 mm Hg and volatiles were collected in a cold trap vacuum (about 10 mL, mostly unreacted SOCl₂). The resulting reaction mixture was used in the next step.

In a separate 1-L, 3-neck round bottom flask in a dry box, was place 45 g of dry TiCl. The flask was placed in a fume hood and equipped with a thermocouple, a heavy magnetic stir bar, a distillation head and an addition funnel. Dry N-ethylpyrrolidinone (NMP, 200 mL) was added to the flask with vigorous agitation to prevent clumping of the LiCl, while the internal temperature was brought up to 50° C. The reaction mixture from the first reaction was placed in an addition funnel and was added slowly to the mixture of LiCl/NMP, while slowly raising the internal temperature to 120° C. over a 6 h period. The product (HCF₂CH₂Cl) was distilled out of the reaction mixture and collected in a receiver, which was cooled with wet ice. The crude product (approximately 75 g) was washed with water (100 mL), dried over MgSO₄ and distilled to give 65 g (64%) of pure HCF₂CH₂Cl, b.p. 32-33 at atmospheric pressure, purity 99% (GC/MS, NMR).

### Preparation of 2,2-Difluoroethyl Acetate

In a dry box, 12 g (0.12 mol) of dried potassium acetate was placed into a 100-mL, 3-neck round bottom flask contain, a heavy magnetic stir bar. The flask was closed with stoppers, removed from the dry box, transferred to a fume hood, and equipped with a thermocouple well, a dry-ice condenser, and an addition funnel under flow of dry nitrogen. Dimethylsulfoxide (DMSO) (50 mL, Aldrich, 99%, 100 ppm of water) was added to the flask using a syringe. Agitation was started and the temperature of the flask contents was brought to about 100° C. HCF₂CH₂Cl (10 g, 0.1 mol) was slowly added to the flask over a 1 h period using an addition funnel. The addition was mildly exothermic and the temperature of the reaction mixture rose to 120-130° C. The reaction mixture was agitated at 120-130 for an additional 6 h (the conversion of chloride at this point was >99%, NMR). Then, the reaction mixture was cooled down and was agitated overnight at ambient temperature. At this point neither the starting chloride nor 2,2-difluorethanol was detectable in the crude reaction mixture by NMR. The dry-ice on the reaction flask was replaced by a hose adapter with a Teflon® valve and the flask was connected to a mechanical vacuum pump through a cold trap (−70° C., dry-ice/acetone). The reaction product was transferred into the cold trap at 40-50° C. under vacuum (1-2 mm Hg, 133 to 266 Pa) to give 12.1 g of HCF₂CH₂OC(O)CH₃ with 96% purity, containing 4% of DMSO. The calculated yield of HCF₂CH₂OC(O)CH₃ was 94%.

### Example 10

### Preparation of 2,2-Difluoroethyl Propionate using HCF₂CH₂Cl in DMSO solvent

2,2-Difluoroethyl propionate was prepared by reacting sodium propionate with HCF₂CH₂Cl in DMSO using the procedure described in Example 9, except that 12 p (0.13 mol) dry C₂H₅C(O)ONa was substituted for the potassium acetate. From the reaction, 13.1 g of C₂H₅C(O)CH₂CF₂H containing 11% of DNSO was isolated. The calculated yield of C₂H₅C(O)CH₂CF₂H was 84.5%.

1. A method of preparing an ester comprising the steps of:
(a) providing a salt of a carboxylic acid represented by the formula:
R¹COO⁻M⁺
wherein R¹ is a C₁ to C₁₀ alkyl group and M⁺ is selected from the group consisting of lithium, sodium, potassium and cesium ion;
(b) providing a fluorinated alkyl halide represented by the formula:
CF₂H—R²—X
wherein R² is a C₁ to C₁₀ alkylene group and X is selected from the group consisting of Br and Cl;
(c) contacting the salt of (a) with the alkyl halide of (b) in a reaction medium comprising a polar, aprotic solvent to form a product that comprises a single ester group; and
(d) optionally, recovering the ester product from the reaction medium.

2.-4. (canceled)

5. The method of claim 1 wherein the fluorine-containing carboxylic acid ester is 2,2-difluoroethyl acetate, 2,2-difluoroethyl propionate, 2,2-difluoroethyl butanoate, or 2,2-difluoroethyl pentanoate.

6. The method of claim 1 wherein the salt of the carboxylic acid is potassium acetate, potassium propionate, potassium butanoate, potassium pentanoate, sodium acetate, sodium propionate, sodium butanoate, sodium pentanoate, cesium acetate, cesium propionate, cesium butanoate, or cesium pentanoate.

7. The method of claim 1 wherein the fluorinated alkyl halide is HCF₂—CH₂—Br or HCF₂—CH₂—Cl.

8.-9. (canceled)

10. The method of claim 1 wherein the polar, aprotic solvent is selected from the group consisting sulfolane, N-methylformamide, N,N-dimethylformamide, N,N-dimethylacetamide, N,N-dimethylpropionamide, dimethylsulfoxide, N-methyl-2-pyrrolidone, N,N-dimethyl-2-imidazolidinone, 1,3-dimethyl-3,4,5,6-tetrahydro-2(1H)-pyrimidinone, and mixtures thereof.

11. The method of claim 1 further comprising the step of admixing the fluorine-containing carboxylic acid ester obtained in step (d) with at least one electrolyte salt to form an electrolyte composition.

12. The method of claim 11 further comprising the step of contacting the electrolyte composition with a cathode and a anode to form an electrochemical cell.

13. The method of claim 12, wherein the electrochemical cell is a lithium ion battery.

14. A fluorine-containing carboxylic acid ester prepared by the method of claim 1.

15. The use of the fluorine-containing carboxylic acid ester prepared by the method of claim 1 in an electrolyte composition.

16. An electrolyte composition comprising the ester of claim 14.

17. An electronic device comprising the electrolyte composition of claim 16.

18. A method of preparing an ester comprising the steps of:
(a) providing a salt of a carboxylic acid represented by the formula:
(R¹COO⁻)ₙM⁺ⁿ
wherein R¹ is a C₁ to C₁₀ alkyl group and M⁺ⁿ is a cation other than hydrogen and n=1 or 2;
(b) providing a fluorinated alkyl compound represented by the formula:
CF₂H—R²—X
wherein R² is a C₁ to C₁₀ alkylene group and X is a leaving group selected from the group consisting of Br, Cl, and —OSO₂R¹⁵ where R¹⁵ is aryl, F, CF₃, C₄F₉, alkyl or OC(O)X where X is Cl or F;
(c) contacting the salt of (a) with the alkyl halide of (b) in the absence of any substance that participates in the formation of an intermediate or reactive substrate from the alkyl halide of (b) to form a product that comprises a single ester group.

19. The method of claim 1 wherein the contacting of step c) occurs in the absence of any substance that participates in the formation of an intermediate or reactive substrate from the alkyl halide of step (b).

20. The method of claim 1 wherein the reaction medium of step c) is substantially free of iodine, iodide ion, chlorine or chloride ion.
